(19) United States
(12) Patent Application Publication
(10) Pub. No.: US 2025/0160688 A1
(43) Pub. Date: May 22, 2025

(54) WEARABLE APPARATUS FOR ANALYZING MOVEMENTS OF A PERSON AND METHOD THEREOF

(71) Applicants: FEATURE JAM S.R.L., TRIESTE (IT); UNIVERSITA' DEGLI STUDI DI TRIESTE, TRIESTE (IT); AZIENDA SANITARIA UNIVERSITARIA GIULIANO ISONTINA, TRIESTE (IT)

(72) Inventors: Andrea PETRONIO, Trieste (IT); Matteo FORMIGLI, Trieste (IT); Walter VANZELLA, Trieste (IT); Milos AJCEVIC, Trieste (IT); Agostino ACCARDO, Trieste (IT); Gianfranco FENU, Trieste (IT); Felice Andrea PELLEGRINO, San Dorligo della Valle (TS) (IT); Eric MEDVET, San Dorligo della Valle (TS) (IT); Giovanni FURLANIS, Trieste (IT); Paolo MANGANOTTI, Trieste (IT)

(73) Assignees: FEATURE JAM S.R.L., TRIESTE (IT); UNIVERSITA' DEGLI STUDI DI TRIESTE, TRIESTE (IT); AZIENDA SANITARIA UNIVERSITARIA GIULIANO ISONTINA, TRIESTE (IT)

(21) Appl. No.: 18/832,516

(22) PCT Filed: Jan. 24, 2023
(86) PCT No.: PCT/IB2023/050593

§ 371 Date: Jul. 24, 2024

(30) Foreign Application Priority Data
Jan. 24, 2022 (IT) 102022000001100

(51) Int. Cl. A61B 5/11; A61B 5/00

(52) U.S. Cl. CPC A61B 5/1123; A61B 5/4839; A61B 5/7264; A61B 5/7275; A61B 5/746; A61B 2562/0219

(57) ABSTRACT

A wearable apparatus for analyzing the movement of a person includes one or more motion detectors for acquiring data related to the movement of specific anatomical districts and a centralized or distributed control system which is configured to execute Artificial Intelligence algorithms for processing movement data. A method for a movement analysis of a person by means of the wearable apparatus is also provided.

### TECHNICAL FIELD

The present invention concerns devices and methods used to analyze movements of a person and particularly to identify anomalous movement patterns with respect to a standard.

### BACKGROUND ART

Systems which are capable of continuously tracking the movement of a person and analyzing relevant features are known in the art. Basically they can be divided into two broad families: “motion tracker” systems are based on motion sensors (e.g. accelerometers), fixed to the body, which generate movement data that are then processed by a control unit to obtain movement information; “motion capture” systems are based on cameras, suitably positioned with respect to the person, which obtains a stream of images that are then processed by a control unit to obtain movement information.

In other words, these systems analyze kinematics of movements, making it possible to obtain useful information i.e., positions gradually assumed by body segments within a predefined volume, or kinematics parameters (i.e., angles, speeds, accelerations and distances) calculation which help to understand in detail a gesture performed by a person.

Movement information and motion tracker/capture systems are widely used in many fields. A noticeable application, is sports activities where they are used, for example, to optimize athletes' performances or improving sport equipment (e.g., sport shoes).

For example, systems of this type are described in www.technogym.com, www.intersense.com, or in the US patents U.S. Pat. No. 8,253,586B1 “Athletic-wear having integral measuring sensors” (in the name of Mayfonk Art Inc), U.S. Pat. No. 8,477,046B2, “Sports telemetry system for collecting performance metrics and data” (in the name of Advanced Technology Co Ltd), U.S. Pat. No. 7,602,301B1 “Apparatus, systems, and methods for gathering and processing biometric and biomechanical data” (in the name of Applied Technology Inc). Finally, systems based on motion tracking for physical rehabilitation and fitness are known for example from “Human motion tracking for rehabilitation—A survey” (DOI:10.1016/j.bspc.2007.09.001). Recently, artificial intelligence (AI) algorithms have been used to classify patterns of movement, e.g., in the patent application CN113762214A “AI artificial intelligence based whole body movement assessment system” in the name of Univ Ningbo. However, it is evident that in CN'214 an AI artificial intelligence system is cited only as a theoretical option and is not fully disclosed in a way which may be useful for the skilled in the art. For instance, the patent specification does not include any citation of specific AI-algorithms neither any description of the way they process movement data in practice.

Therefore, CN'214 is not useful to those skilled in the art wishing to take advantage of AI-based algorithms for body movement assessment.

More relevant teachings related to the use of artificial intelligence or machine learning to movement analysis are disclosed in the US applications US2017188895A1 and US2017196497A1.

The application US'895 “System and method of body motion analytics recognition and alerting” in the name of Smart Monitor Corp. US'895 may be considered as the closest prior art document considering that it includes many technical features of the present invention: body-worn sensors and an AI-based control unit to process movement data and identify movement patterns. However, the method and system for continuous patient monitoring of US'895 are based on a centralized processing unit. In this architecture the centralized processing unit has to process the movement data streams generated by all the body-worn sensors. To increase efficiency in anomalies detection, acquisition frequency of movement data must be increased. This results in a huge data load which stresses communication channels and computing capability of the centralized processing unit. Therefore, an improved architecture is needed. However, US'895 does not discloses, nor suggests, how to perform body motion analytics by means of a more efficient architecture, e.g., a distributed processing architecture where body-worn sensors are involved in the pattern analysis.

For the purpose of assessing novelty of the present invention, another relevant document is the US application US'497 “System and method for identifying ictal states in a patient”, in the name of Dartmouth College. US'497 discloses a continuously wearable device useful to detect and record epileptic seizures during normal patient routines. The device uses a plurality of extra-cerebral sensor modalities coupled with a responsiveness test.

Although the responsiveness test is based on machine learning and feature vectors derived from the plurality of sensors, US'497 is specifically addressed to ictal states identification, i.e., to people suffering from epilepsy.

Therefore, it is not useful to those skilled in the art wishing to analyze movement data to identify anomalies which are not related to, or predictive of, epilepsy seizures.

Furthermore, US'497 lacks to describe a distributed architecture of the processing unit. Despite improvements provided by US'895 and US'497, known systems still lack to efficiently recognize (i.e., minimize false positives) anomalies in the movement of a person according to the best knowledge of the present inventors.

The term “movement anomaly” or “anomaly in the movement” may assume different meanings depending on the field where the motion tracker device (or motion capture device) is used. If the person whose movement has to be analyzed is an athlete, particularly a professional athlete, a movement anomaly is an athletic gesture that differs from the “best practice” i.e., the best performance of said athlete or gestures executed by best players.

In case the person is a patient recovering from an injury through rehabilitation assistance, a movement anomaly is a gesture that differs from the one suggested by the rehabilitation trainer e.g., the movement to be done to accelerate post-trauma recovery or to retrain a limb in post-trauma motor retraining.

In case of a person affected (or presumed to be affected) by a pathology, the movement anomaly is the result of the pathology itself. In fact, as confirmed by a number of clinical studies, neurological pathologies are associated with an alteration of the movement pattern. Among the neurological pathologies, stroke and Parkinson's disease are the most remarkable examples. Motor deficit, and in particular paresis of the limb contralateral to the site of the brain lesion, is one of the most common signs caused by a stroke.

According to the World Health Organization (WHO), stroke is the sudden onset of symptoms referable to local and/or global deficit (coma) of brain functions, which lasts for more than 24 hours (if not death of the patient). Stroke produces persistent damages to the brain having vascular origin. In Western countries, cerebral stroke represents the third cause of death, after cardiovascular diseases and cancer, and the absolute first cause of disability. Furthermore, stroke is the second leading cause of dementia.

According to the Società Italiana di Neurologia, globally around 15 million people are affected by stroke each year; among these 5 million face death and another 5 million permanent disabilities. In European countries the incidence of stroke varies between 95 and 290 new cases per 100,000 inhabitants per year and every year there are 650.000 deaths caused by stroke. In Italy stroke has an incidence of about 9 per thousand in the population aged 64-84 which corresponds to a about 200,000 new cases per year, of which 80% are first events and 20% are relapses. Motor impairment accounts for 85% of patients in the acute phase and persists completely or partially in about 50% of patients who survive the acute event.

On the basis of the generation mechanism, cerebral stroke can be divided into ischemic stroke (85% of cases) and hemorrhagic stroke (15%). Ischemic stroke is caused by a blood clot blocking, or reducing, blood flow to the brain. If the cerebral blood flow is interrupted only for a very short period, and normal blood flow is re-established, the person faces a Transitory Ischemic Attack (in short TIA). In fact, TIA is defined as the sudden onset of signs and/or symptoms referable to focal cerebral or visual impairment determined by an insufficient blood supply lasting less than 24 hours (in most cases less than 1 hour) without evidence of a cerebral infarction. An estimated 20%-25% of ischemic strokes are preceded by a TIA.

With effective reperfusion therapies, ischemic stroke is potentially treatable in the very first hours after the onset of symptoms. After years of tremendous efforts to understand the mechanisms of brain damage, the most effective therapy in the pharmacopoeia is based on the recombinant thrombolytic agent named as “tissue plasminogen activator” (rTPA). Recently, mechanical thrombectomy using stent retrievers and thromboaspiration catheters has joined intravenous thrombolysis as an effective treatment opportunity in selected individuals.

These therapies can be offered to the patient only within a strictly defined time window. Therefore, the timely recognition of symptoms is essential to allow the application of therapies and increase the probability of a favorable outcome, minimizing motor deficits.

With 85% of patients in the acute phase, motor deficit is the most present symptoms of ischemic stroke. Therefore, a device capable of promptly detecting the onset of a motor deficit could improve safety especially in the population with numerous vascular risk factors or unable to promptly alert emergency services, both within hospital facilities and at home.

Furthermore, an objective assessment of the motor deficit could be useful for assessing motor recovery in the post-acute phase and during rehabilitation.

However, systems capable detecting movement anomalies associated with pathologies are still not available because of the high false positive rate despite the use of machine learning technology to increase efficiency.

To conclude, the problem of producing an apparatus and implementing a method for efficiently identifying anomalies in the movement of a person, has not been completely addressed in the state-of-the-art. Said apparatus and method would be useful in many fields e.g., in professional sports business to optimize athletes' performance, in motor rehabilitation to accelerate post-trauma recovery, as well as in early prediction of pathologies in particular of stroke or TIA.

### DISCLOSURE OF INVENTION

### Object of the Invention

It is therefore needed a wearable apparatus and a method for analyzing movements of a person, and particularly for identifying anomalies in the movement which are not affected by the limitations and drawbacks cited above.

Accordingly, the first and main object of the present invention is providing an apparatus and a method thereof for identifying anomalies in the movement of a person with respect to a defined standard movement of that person. This purpose includes identification of movement abnormalities in sports activity, post-trauma rehabilitation or in early-stage prediction of diseases having an impact on the way a person moves (e.g., a motor deficit), in particular a stroke or a TIA.

A second object of the present invention is providing an apparatus for identifying movement anomalies which can be worn by the person and which can be easily fitted and integrated into existing wearable systems.

A third object of the present invention is providing said apparatus and related method which includes means and functions for activating an alarm when a movement abnormality is validly detected.

A fourth important object of the present invention is to develop said method in a way which minimizes false positives and therefore can effectively distinguish movement anomalies actually associated with symptomatic events of an existing or early-stage pathology.

A fifth object of the present invention is providing said apparatus and related method which can administer a composition to counteract or mitigate the effects of the condition associated with the movement anomaly, e.g., in case of a stroke or a TIA, a life-saving drug.

Finally, a final object of the present invention is to provide an apparatus for identifying anomalies in the movement of a person and a method thereof, which can be produced or enabled in a simple way and by means of possible low-cost technologies.

### Technical Solution

These and still other objects, which will appear more clearly hereinafter, are achieved by an apparatus for wearable apparatus for analyzing the movement of a person and a method thereof. The invention is defined by the appended independent claims 1 and 11 while advantageous features are set forth in the appended dependent claims. The aforesaid claims, to which reference should be made for the sake of brevity, are hereinafter specifically defined and are intended as an integral part of the present description.

The present patent discloses a wearable apparatus and a method for analyzing the movement of a person, and particularly for identifying anomalies in said movement.

For the sake of clarity, the term “anomaly”, singular or plural, referred to a movement of the person shall mean movement of an anatomical district which is not consistent with a standard condition of the person. By way of explanation of the invention, and not as a limitation thereof movement anomalies include: an athletic movement of an anatomic district differing from best practices or performances, gesture differing from the one suggested by the rehabilitation trainer to accelerate post-trauma recovery, incorrect movements in post-trauma motor retraining, motor deficits or impairments related to, or predictive of, a pathology e.g., a stroke or a TIA.

The wearable apparatus comprises one or more wearable devices positioned on the anatomical districts of the upper and lower limbs, preferably at least one per district. Optionally said apparatus includes one or more additional devices positioned on the head, neck, trunk and pelvis. For example, an apparatus may comprise two wearable devices, the first fixed to an arm (or forearm, elbow, wrist, hand, fingers), the second on the thigh (or knees, leg, ankle, foot, fingers).

Each wearable device includes one or more motion detectors for acquiring data related to the movement of the anatomical districts. Detectors generate an output in the form of a “raw” data flow of the speed, acceleration or position of the anatomical districts.

Each wearable device is enclosed in a casing having fixing means for positioning the device to the anatomical region.

The wearable apparatus according to the invention comprises a control unit to which the wearable devices are functionally connected via a communication module. Optionally the control unit may include one or more remote electronic devices i.e., an computer or a notebook. Anyhow, the control unit comprises a processor and a memory unit having a computer program stored therein. The control unit is configured to execute Artificial Intelligence (AI) algorithms for processing the movement data flow detected by the wearable devices.

The wearable apparatus includes also a user interface which may be integrated to the master device or to the remote control unit.

Finally, the apparatus may optionally include administration means allowing the automatic or manual assisted administration of a composition, e.g., a life-saving drug.

Compared to known technologies, the wearable devices and AI algorithms are able to exchange movement data and processed movement data with different sharing level.

In fact, the control unit and the AI algorithms have a distributed or a centralized architecture. Particularly, in the wearable apparatus according to the present invention there are a master device and one or more slave devices. A master device receives data flows from all the motion detectors and performs a first classification or a further classification in addition to those performed locally by individual devices. The other devices of the apparatus are defined as slave devices.

The apparatus and method according to the present invention are configured in a way that the flow of movement data can be read in real time (or in near real time) by the control unit which can process and share them in different ways with the other devices.

In one embodiment, each device can autonomously and independently detect an anomaly in the movement of an anatomical district and provide a classification in real-time. If a significant event is detected, the device activates an alarm signal.

In an alternative embodiment, the control unit has a centralized architecture where a single device performs decision-making tasks. The single device can be the master device or alternatively, the remote control unit. If a significant event is detected, the master device or the remote control unit activates an alarm signal.

This peculiar architecture, enables an “AI-embedded” paradigm, which advantageously, improves detection rate, even in real time, of potential anomalies in the movement data respect to a defined standard or normal condition of the person.

By involving in the decision process a plurality of devices, it is thus possible to improve the reliability of the classification of the anomalous event with greater confidence.

The apparatus according to the present invention is useful during post-trauma motor rehabilitation (e.g., to improve recovery) or during sport training sessions (e.g., to optimize athletes' performance) as well as when the movement anomalies are related to, or predictive of, pathologies (e.g., a stroke or a transitory ischemic attack).

In the latter case, the apparatus can detect the fall of a person at risk of TIA or ictus. In this way, emergency services can be alerted timely, especially when caregivers or medical staff are not available on site, thus increasing chance of recovering neurological functions otherwise irreversibly compromised by the ictus.

### BRIEF DESCRIPTION OF DRAWINGS

The features and advantages of the present invention will be more fully understood by reference to the following drawings which are provided solely for illustration of the embodiments and not limitation thereof:

FIG. 1 schematically illustrates the apparatus according to the present invention;

FIG. 2 schematically illustrates a device of the apparatus of FIG. 1;

FIG. 3 schematically illustrates the functional diagram of the apparatus of FIG. 1 where the devices of FIG. 2 exchange movement data with a total sharing level to detect an anomaly in the movement of the person

FIG. 4 schematically illustrates the functional diagram of the apparatus of FIG. 1 where the devices of FIG. 2 exchange movement data with a partial sharing level to detect an anomaly in the movement of the person;

FIG. 5 schematically illustrates the functional diagram of the apparatus of FIG. 1 where the devices of FIG. 2 exchange movement data with a minimum sharing level to detect an anomaly in the movement of the person;

FIG. 6 schematically illustrates the apparatus according to the first preferred embodiment of the present invention;

FIG. 7 schematically illustrates the apparatus according to the second preferred embodiment of the present invention;

FIG. 8 schematically illustrates the apparatus according to the third preferred embodiment of the present invention.

These figures illustrate and demonstrate various features and embodiments of the present invention but are not to be construed as limiting the invention.

### DETAILED DESCRIPTION OF THE INVENTION

Wearable apparatus for analyzing the movement of a person A first object of the present invention is a wearable apparatus (2) for analyzing the movement of a person (P) having the characteristics defined in the appended claim 1, which is intended as an integral part of the present specification but it is omitted here for the sake of brevity.

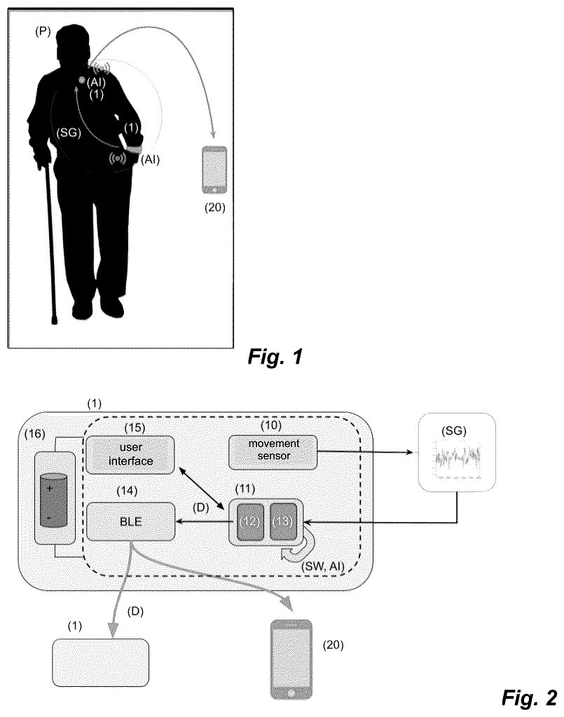

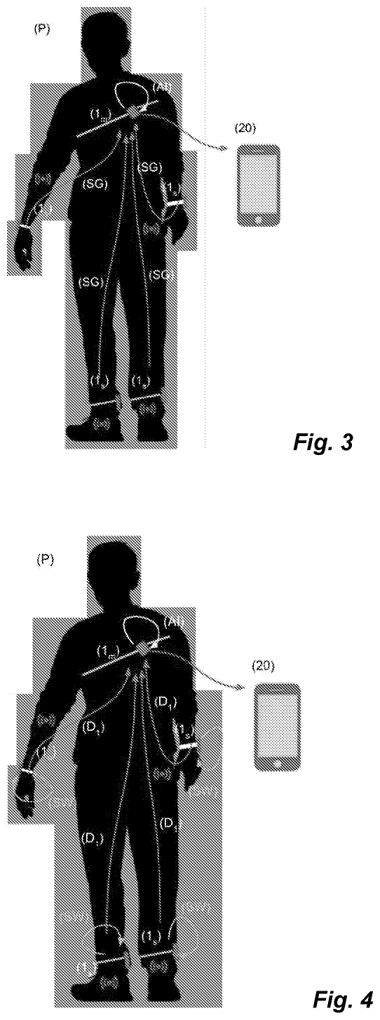

In summary, with reference to FIG. 1, said apparatus (2) is characterized by the fact of including: one or more devices (1) for acquiring and optionally processing data related to the movement of one or more anatomical districts (AD) of a person (P); a centralized or distributed control unit (11) comprising a memory unit (13) in which a computer program (SW) executable by a processor (12) is stored; a communication module (14) for movement data exchange between two or more of said devices (1) or between at least one of said devices (1) and a remote electronic device (20).

The wearable apparatus (2) is also characterized in that the computer program (SW) includes one or more artificial intelligence algorithms (AI). Particularly, in association with the processor (12) and the communication module (14), they process or analyze, according to an “AI-embedded” paradigm, data related to the movement of anatomical districts (AD) in order to identify, even in real time, potential anomalies in the movement of the person (P) with respect to a defined standard or normal condition.

Components and functions of the wearable apparatus (2) are described in detail below by way of explanation of the invention, and not as a limitation thereof.

The wearable apparatus (2) according to the invention includes one or more devices (1) for tracking the movement. In turn, each of said devices (1), which are schematically illustrated in FIG. 2, comprises one or more motion detectors (10) to track the movement of the anatomical district (AD) carrying the device (1). Preferably, each device (1) includes a single detector (10). However, for the purposes of implementing the present invention, wearable devices (1) with a different number of detectors (10) can also be used.

Said detectors (10) detect and record physical entities directly associated with a movement, (e.g., speed, linear acceleration, angular acceleration) by means of accelerometers, gyroscopes and inertial type sensors, or other equivalent sensors.

For the purposes of implementing the present invention, it is also possible to use detectors (10) able to detect and record entities indirectly related to a movement (e.g., position of a person, magnetic field) by means of magnetometers or cameras. Appropriate processing of acquired images or videos is required to extract movement data.

Anyhow, detectors (10) generate an output in the form of a “raw” data flow (SG) of the speed, acceleration or position of the anatomical districts (AD). The stream (SG) can be read in real time, or in near real time, by the control unit (11) which can process and share them in different ways with the other devices (1) as it will be explained below.

The wearable apparatus (2) according to the invention comprises a control unit (11) to which the devices (1), in particular the motion detectors (10), and optionally one or more remote electronic devices (20) are functionally connected. With reference to the enclosed FIG. 2, said control unit (11) comprises a processor (12) and a memory unit (13), where a computer program (SW) executable by said processor (12) is stored. The processor (12) is able to process and/or transmit a data flow (SG) related to the movement parameters, i.e., speed, acceleration or position of the anatomical districts (AD) where devices (1) are applied. An output consisting of one or more processed datasets (D) is produced at the end of the data flow (SG) processing.

For the purposes of implementing the present invention, the processor (12) and the memory unit (13) are of known type. A low-energy processor (12) is the preferable option.

In one embodiment, the memory unit (13) comprises a high-speed main memory (131) and a mass memory (132). This configuration makes it possible to save the data flow (SG) generated by the detectors (10) in the main memory (131), when it is preferable that processing for detecting a movement anomaly is performed locally in real time. Alternatively, the data flow (SG) is saved in the mass memory (132) when it is possible, or desirable, that processing of the data flow is performed in deferred mode by a different device (1) or by an external device (20).

As mentioned, a computer program (SW) is stored in the memory unit (13) which can execute one or more artificial intelligence algorithms to perform the following tasks: first, identifying potential anomalies in the movement of the person (P) from the data flow (SG) generated by the detectors (10); then, evaluating those anomalies which are actually associated with, or predictive of, an anomalous event of the person (P), e.g., the onset of a pathological condition. The computer program (SW) and the artificial intelligence algorithms will be described later. In one embodiment, the control unit (11) is functionally connected only to the devices (1). In this case, the data flow (SG) is processed by one or more of these devices (1) according to methods which will be explained later.

In an alternative embodiment, the control unit (11) is functionally connected to the devices (1) and in addition to a remote electronic device (20). Preferably the remote electronic device (20) has an external memory unit (201). Examples of remote electronic devices include: smart-phones, tablets, personal computers, a gateway, video game consoles (combinations of such devices are also possible). In said embodiment, the data flow (SG) is processed by the remote electronic device (20), preferably a PC, and optionally by one or more of the devices (1) according to methods which will be explained below following.

Combinations between such embodiments are lo possible.

For greater clarity, it should be noted that the term “functionally connected”, referring to the relationship between the components (1,20) of the wearable apparatus (2), designates a physical or wireless connection which determines a one-way or two-way data exchange between the components involved in the connection.

The control unit (11) may have both a distributed or a centralized architecture. Exemplary embodiments of apparatus architectures are disclosed in the following by way of explanation of the invention, and not as a limitation thereof.

In one embodiment, thanks to distributed artificial intelligence (AI) algorithms, each device (1) can autonomously and independently detect an anomaly in the movement of an anatomical district (AD) and provide a classification in real-time. If a significant event is detected, the device (1) activates an alarm.

In an alternative embodiment, the control unit (11) has a centralized architecture where a single device (1) performs decision-making tasks. Said device executes artificial intelligence (AI) algorithms to the data outputted from all the other devices (1) to improve the classification process or to identify a significant event with greater confidence. In a centralized architecture, the processing mode can be in real-time or in deferred mode.

Hybrid modes are also possible by combining the previous embodiments.

Further details on the various architectures of the apparatus (2) according to the invention will be provided in the following.

The wearable apparatus (2) according to the invention comprises a communication module (14) which is connected to the processing unit (11). In one embodiment, the communication module (14) is integrated, at least partially, with the device (1) for tracking movement. The main function of this module (14) is twofold. Firstly, it allows data exchange between wearable devices (1) and a remote electronic device (20) if it is involved in the movement data analysis. Furthermore, it guarantees connectivity of the device (1) to a remote electronic device (20), e.g., via Internet or a telephone network, to enable alarm activation in case a significative anomaly in the movement (e.g., an incorrect limb movement in motor reprogramming) is detected.

For the purposes of implementing the present invention, it is preferable that the communication module (14) is of the type Bluetooth® Low Energy, in short BLE (Bluetooth® is a trademark of Bluetooth Special Interest Group Inc). In fact, this technology allows a considerable reduction in energy consumption and at the same time ensures a data transmission speed that is adequate for the device and apparatus according to the present invention.

Alternatively, other known types of wireless communication modules and protocols can be used, provided that they allow data transmission between the control unit (11) and the motion detectors (10) within 10 meters, preferably within 3 meters. Hard-wired communication, for example by channel/bus, such as 12C or SPI may also be used.

Anyhow, data exchanged through the communication module (14) are the data flow (SG) generated by the detectors (10) or the datasets processed (D) by the control unit (11).

From the description provided, it will be apparent to those skilled in the art that by combining the control unit (11) and the communication module (14), different architectures can be obtained for the wearable apparatus (2) according to the present invention.

For the sake of clarity, in the architectures which are disclosed in the following, devices (1) can be subdivided in a “master device” (1ₘ) and “slave devices” (1ₛ).

A master device (1ₘ) receives data flows (synchronized and therefore associated with a common time frame) from all the motion detectors (10) and performs a first classification or a further classification in addition to those performed locally by individual devices (1). The other devices (1) of the apparatus (2) are defined as “slave devices” (1ₛ).

In one embodiment, the “master” device (1ₘ) is one of the wearable devices (1).

In a further embodiment, the “master” device (1ₘ) is a remote electronic device (20) having an external memory unit (201).

These architectures correspond to different methods of processing the stream of data generated by motion detectors (10) which differ from each other in the way they share the tasks for identifying anomalies in the movement.

For example, in one embodiment, the analysis of movement data can be done by individually considering all the signals generated or processed by each single device (1).

In an alternative embodiment, all the synchronized signals coming from each single device (1) are processed simultaneously. Combinations of two embodiments are also possible.

The wearable apparatus (2) according to the invention comprises a user interface (15), which in association with said software (SW) allows to set and activate the functions of said apparatus (2) particularly an alarm condition. The user interface (15) includes an alarm device (151), such as an audible alarm, LED devices, or a vibrating motor, and an interaction device (152) between the user and the device, e.g., command keys or a touch display. By means of specific light and sound patterns, the user interface (15) can signal relevant information related to the person (P) and derived from the analysis of the movement anomaly of specific anatomical districts (AD). The user interface (15) can also manage error conditions related to malfunction of the device (1) or lack of connection to remote electronic devices (20), e.g., due to absence of Internet connection.

By way of explanation of the invention, and not as a limitation thereof, in one embodiment, the user interface (15) is integrated in the master device (1ₘ).

In an alternative embodiment it is integrated in the remote device (20), preferably in a smart-phone. Further embodiments are possible according to needs.

The wearable apparatus (2) includes a centralized or distributed power supply device (16). In one embodiment, the power device (16) comprises one or more batteries (161) and a battery manager (162) which manages their charge status. In the design stage of the apparatus (2) the present inventors tried to achieve a trade-off between consumption reduction and high performance in movement anomalies identification. Consequently, each power supply device (16) preferably includes a low energy consumption battery, rechargeable or non-rechargeable, which can power the apparatus (2) for a minimum of 24h up to a whole week.

In an alternative embodiment, the power device (16) can be different e.g., an energy harvesting device.

As will be explained in detail below, duration of the power device (16) is determined by a number of parameters mainly: real-time or deferred processing mode; sampling frequency; communication load between the device (1ₘ) and remote electronic devices (20), or other devices (1ₛ); complexity of the algorithms executed by the processor (12) which vary according to the specific application of the device (1).

By way of explanation of the invention, and not limitation thereof, if the analysis of movement anomalies is addressed to recognize events associated with pathologies, such as a TIA or a stroke, the capacity of the battery (131) must guarantee a duration in order of magnitude of the period in which the patient is considered to be at high risk for a stroke, about 1-10 days.

In one embodiment, the wearable apparatus (2) includes a centralized power supply (16) equipped with a single battery (161) which power all devices (1) and the other connected components i.e., the motion detectors (10), the processing unit (11), the processor (12), the memory unit (13), the data transmission module (14) and, if present, the user interface (15).

In an alternative embodiment, the wearable apparatus (2) includes a distributed power device (16), where each device (1) is independent from the others as regards the power supply. Combinations of centralized vs. distributed architectures of the power system (16) are possible. The wearable apparatus (2) according to the invention comprises one or more wearable casings (17) which contain one or more devices (1) and at least a part of the communication module (14). However, based on the architecture of the wearable apparatus (2) according to the invention, the casing (17), or casings, may contain other components such as the control unit (11), the power supply device (16) and the user interface (15).

In one embodiment, the wearable apparatus (2) includes a casing (17) for each of the devices (1). This casing (17) can also include other components such as the control unit (11), the communication module (14) and the supply device (16). In this embodiment, thanks to suitable fixing means, for example a velcro closure, the casing (17) holds the device (1) on a specific anatomical region (AD), making it possible to track motion of this region.

In an alternative embodiment, the wearable apparatus (2) includes a casing (17) which makes all the devices (1) wearable. In this case, the casing (17) may take the form, for example, of one or more pockets fixed to a jacket or pair of trousers. Pockets allow positioning of the devices (1) in correspondence of each anatomical district (AD) whose movement is to be monitored. In this way, it is possible to ensure maximum flexibility in positioning and movements tracking of any part of the body.

Finally, the wearable apparatus (2) for analyzing the movement of a person (P) according to the invention can optionally include an administration means (18) which is configured to automatically or manually administrate a compound when the control unit (11) detects an anomaly in the movement related to, or predictive of, a pathological condition.

In one embodiment said compound is a drug which is administered on the basis of a predefined therapy protocol. Drug can be supplied automatically, i.e., in case the wearable apparatus (2) detects an event predictive of a pathological condition, and also manually in case the patient (or a family member) recognizes the symptoms of his/her disease.

However, other activation methods are possible.

The administration means (18) of the wearable apparatus (2) according to the invention is of known type, for example it can be the one used in the automatic administration of insulin in diabetic patients.

### Method for a Movement Analysis of a Person

The wearable apparatus according to the invention is useful to implement a method for identifying anomalies in the movement of a person (P) with high efficiency and possibly in real time. By way of explanation of the invention, and not as a limitation thereof, anomalies may be related to a pathological condition e.g., a TIA or a stroke.

Therefore, a further object of the present invention is a method for a movement analysis of a person (P) by means of the wearable apparatus (2) described above. Said method synthetically includes the following steps:
a) Selection and positioning of the wearable device;
b) Initialization and training of the apparatus;
c) Movement data acquisition;
d) Movement data processing;
e) Movement data classification and anomaly identification;
f) Alarm activation.

Detectors (10) of the wearable devices (1ₘ, 1ₛ) generate signals in the form of a data flow (SG) which is read in real time by the processor (12) of the local or distributed processing unit (10).

This in turn can process and manage data in different ways.

In one embodiment, the data flow is stored in the main memory (221) or in the mass memory (222) of the device (1ₘ, 1ₛ). Each device (1ₘ, 1ₛ) processes in real-time the stream at a local level before delivering the output to the master device (1ₘ) via the communication module (14). This embodiment is preferable if real-time processing of the data is required. When a pre-defined number of signal samples is achieved, these are processed by the processor (12), which is part of a distributed control unit (10) and hence associated with all the devices (1).

Processing involves the use of classification algorithms (AI) or signal encoding algorithms implemented by a software (SW).

In an alternative embodiment, the data flow is stored in the mass memory (222) of the device (1ₘ, 1ₛ). In this embodiment, the mass memory (132) stores the raw signal (SG), or the sampled signal (D₁), which is possibly processed by the processor (12) in differed mode. In this configuration also, the processor (12) is part of a distributed control unit (10) and hence it is associated with all the devices (1). This mode allows to keep on board the device (1ₘ, 1ₛ) data that can be written by the processor (12) during monitoring of the person (P). For example, the memory (132) allows the processor (12) to keep track of several quantities/parameters obtained by signals measured by detectors (10) which in turn enables evaluation of the state of the person (P), such as mobility, when wearing the wearable device (2).

In still another embodiment, the data flow is immediately sent by the slave devices (1ₛ) to the master device (1ₘ) via the communication module (14) without local processing. In this embodiment, the raw signal (SG), or the sampled signal (D₁), is stored in the mass memory (132) of the master device (1ₘ) and is then processed by the processor (12), which is part of a centralized control unit (10) and hence associated with the single master device (1ₘ).

In a further embodiment the data flow, via the communication module (14), is immediately sent by the slave devices (1ₛ) to a remote device (20) which performs the function of master device (1ₘ). In this form, the raw signal (SG), or the sampled signal (D₁), is stored in the external memory unit (201) which is possibly processed by the processor (12) of the remote device (20) in differed mode.

The last two embodiments require that the remote device (20) and the module (14) support a compatible communication protocol. Preferably the protocol is wireless Bluetooth® Low Energy (BLE). However, other protocols can also be used in wired mode.

In any case, disclosed procedures are based on a family of classifying algorithms (AI) that operate according to the machine learning (ML) paradigm.

More in detail, the classifier algorithms (AI) associate a binary output (detected event/undetected event) to a signal time frame, or to a “vector features” (or simply “features”), i.e., a representation extracted from signal time frame. The possibly extraction of the features is part of signal pre-processing.

Depending on the selected architecture, the classification is performed independently by each slave device (1ₛ) only on the signals it collects and possibly pre-processes. Alternatively, the classification is performed by the master device (1ₘ) on the set consisting of all the signals collected or on the set made of the vector features. In fact, as it will be explained in detail below, wearable devices (1) can exchange movement data with different sharing level.

Each classifier (AI) implemented in the master device (1ₘ) or in the slave devices (1ₛ) consists of a set of heterogeneous classifiers also named “ensemble”. By operating independently on the same signal window, or on the its vector features, each heterogeneous classifier provides a distinct output. The output of the ensemble is the dataset (D₃), which represents the output of the classification stage. It is a function of the output of the individual classifiers of the ensemble i.e., the outcome of a majority vote, possibly weighted.

The individual classifiers which constitute the ensemble, are classifiers known in the art. Further details of the process are provided below by way of explanation and not limitation of the present invention.

### a) Selection and Positioning of the Wearable Device.

The method according to the invention starts by selecting a wearable apparatus (2) having a pre-defined number of wearable devices (1) such as those previously described. These devices are suitably positioned on at least one anatomical district (AD) of the person (P) whose motion is to be analyzed when he moves.

The choice of the type of wearable device (2), particularly the number and position of said devices (1), depends on the physical condition or the presumed anomalous or pathological condition of the person (P) to be monitored and detected.

In one embodiment, the person (P) is an athlete wishing to optimize performance by analyzing movements of certain anatomical districts during training sessions.

In an alternative embodiment, the person (P) performs rehabilitation sessions following a trauma that affected movements of certain anatomical districts (AD).

In a further embodiment, the person (P) is affected, or is presumed to be affected, by a pathological condition causing motion deficits which, for example, affect limbs.

By way of explanation and not limitation of the present invention, pathological conditions yielding motion anomalies or deficits includes: TIA, stroke, epilepsy, Asperger's syndrome, autism, or a combination thereof.

### b) Initialization and Training of the Apparatus.

In this step, training of the classifier algorithms (AI) is performed according to a typical machine learning approach. Training involves providing to an ensemble of classifying algorithms (AI) controlled samples of both “normal” or “abnormal” movement sequences (i.e., related to anomalous or pathological conditions) of the person (P).

Taking advantages of the controlled samples provided, algorithms are trained to recognize in the sequences certain “general” patterns of anomalous movement (AMP) that may be related to an anomalous condition. In summary, capability of the system to distinguish anomalous patterns in general movement sequences is obtained from controlled and specific samples.

The ensemble includes one or more classifying algorithms (AI). By way of explanation and not limitation of the present invention, the individual classifiers include (see bibliographic references given below): recurrent neural networks″^{[2]}, convolutional neural networks^{[2]}, one-dimensional convolutional neural networks″^{[3]}, random forests^{[4]}, support vector machines^{[5]},″ k-nearest neighbor^{[6]}.

In one embodiment, the classifier algorithms (AI) are trained to recognize a “standard” movement of the person (P) by providing said ensemble with a set of normal movement patterns (NMP).

In an alternative embodiment, said classifying algorithms (AI) are trained to recognize an anomalous movement (“anomaly detection”) by providing said ensemble with a set of anomalous movement patterns (AMP) associated with movement anomalies of the person (P). However, combinations of these embodiments are possible to recognize normal movement patterns (MPN) from anomalous movement patterns (AMP) and therefore to distinguish a “standard” behavior from an anomalous one.

To increase classification performance and minimize false negative outcomes, training of the classifier algorithms (AI) of the wearable apparatus (2) is customized and requires definition of a standard in a number of conditions since each person (P) has own definable as “normal”. In one embodiment, the method for analyzing the movement of a person (P) by means of the apparatus according to the present invention is used to identify anomalies in the movement predictive of, or associated with, a human pathological condition. In this case, movement data from people having motion deficits, e.g., people hospitalized following a stroke, can be used if movement data from people having a stroke are not available. Abnormal movement patterns (AMP) characterize events such as stroke and/or TIA, in which one or more limbs, whose movement are monitored, are affected by a plegia or an immobilization condition.

### c) Motion Data Acquisition

In this step, movement data flow (SG) generated by the motion detectors (10) is sampled at a given frequency to output a first dataset (D₁). Said frequency is preferably 100 Hz.

By way of explanation and not limitation of the present invention, the first dataset (D₁) has a representation of the type {SG₁, SG₂, . . . SGⱼ}, where SG; is the data flow generated by the generic sensor (10) with 1≤j≤J (J is the total number of detectors of the wearable device).

By way of explanation and not limitation of the present invention, the first dataset (D₁) has a representation such as {D_{1, 1}, D_{1, 2}, . . . D_{1, 1}}, where D_{i,j} designates data windows, with or without overlap, obtained from the generic sensor with j con 1≤j≤J.

### d) Motion Data Processing.

Signals acquired and sampled in the previous step are suitably processed before being classified by the algorithms (AI). A processing operator is applied to the first dataset (D₁) to obtain a second dataset (D₂).

In one embodiment, the operator is a time-shift synchronization operator which operates a time translation of the elements in the first dataset (D₁) generated by different devices (1), so that they refer to the same time frame. In this way, by analyzing at the same time signals (D₁) from different devices (1), the operator increases precision in movement anomaly identification and hence prompt activation of an alarm condition.

In an alternative embodiment, the processing operator applied to the first dataset (D₁) extracts features i.e., characteristic portions of the sampled movement sequences. In practice, the operator provides a representation of the first dataset (D₁) in the form of a “feature vector”, where each element of said dataset is replaced by one or more corresponding “features”.

In still another embodiment, the operator is executed directly in the device (1ₘ, 1ₛ) which generated the signals to obtain a pre-processed data flow. This mode reduces the load on the transmission channels, e.g., by eliminating irrelevant portions from the sampled sequences. It will be apparent to those skilled in the art that further embodiments can be obtained by combining the previous ones. For example, extraction of the “features” from the movement sequences may be done after the time-shift synchronization operator is executed when signals (D₁) from different devices (1ₘ, 1ₛ) are analyzed at the same time.

### e) Motion Data Classification and Anomaly Identification.

A classification procedure including the classification algorithms (AI) is applied to the second dataset D₂ obtained at the end of the previous step, in order to generate a third dataset (D₃). This dataset expresses the degree of similarity between the movement of the person (P) associated with the data flow (SG) previously acquired, and the set of normal movement patterns (NMP) or the set of anomalous movement patterns (AMP). Therefore, it allows identification of those motion anomalies having a significant probability of being incorrect or potentially related to an abnormal or pathological condition, for example a stroke/TIA event. In the classification procedure, a metric is used to evaluate the “distance” between the anomaly in the movement of a certain anatomical district (AD) and a standard condition of the person (P). The metric is based on both the set of normal movement patterns (NMP) and the set of anomalous movement patterns (AMP) defined at the beginning of the process according to the present invention. For the purposes of implementing the present invention metrics of a known type can be used.

By way of explanation and not limitation of the present invention, motion anomalies include: deviation from the optimal movement of an athlete; an incorrect movement during a rehabilitation session; fall of the person; absence of movement related to a fainting/loss of consciousness which may be the result of a stroke/TIA event.

In one embodiment, the classifier algorithms (AI) are executed by the individual wearable devices (1ₘ, 1ₛ) so that the classification step is performed locally independently by each device (1ₘ, 1ₛ). In this mode, the classification step involves only signals collected, sampled and processed by each device (1ₘ, 1ₛ) and therefore only elements of the first dataset (D₁) or of the second dataset (D₂). In other words, each device (1ₘ, 1ₛ) determines any movement anomaly independently following a “local” classification process. The master device (1ₘ) determines any anomalies by giving a more or less relevant weight to each device slave (1ₛ) based on the data sharing level between the devices (1ₘ, 1ₛ).

In an alternative embodiment, the classifying algorithms (AI) are executed only by the master device (1ₘ) so that the classification step is performed centrally. In this mode, the classification step involves all the signals received from the slave devices (1ₛ), and therefore the entire first dataset (D₁) or the entire second dataset (D₂). In other words, only the master device (1ₘ) determines any anomaly in the movement according to a “centralized” classification process. However, combinations of such embodiments are also possible.

It will be evident to those skilled in the art that in this step of the process according to the present invention the classification of the movement anomaly depends on the architecture of the wearable apparatus (2). As mentioned above, the architecture determines different degrees of data sharing between devices (1ₘ, 1ₛ, 20).

In one embodiment, the sharing level between said devices is total. In this mode, the first dataset (D₁) is generated by each device (1) and is sent to the single master device (1ₘ, 20) which processes it and operates a classification by generating, respectively, a second dataset (D₂) and a third dataset (D₃).

In an alternative embodiment, the sharing level is partial. In this mode, each slave device (1ₛ) performs locally data processing, and generates a second dataset (D₂) which is shared with the master device (1ₘ, 20). In practice, each slave device (1ₛ) shares with the master device (1ₘ) only D₂ signals already processed locally.

In a further embodiment, the sharing level is minimal. In this mode, each slave device (1ₛ) generates a second dataset (D₂) and a third dataset (D₃), but each slave device (1ₛ) shares with the master device (1ₘ, 20) only the third dataset (D₃), i.e., the result of the classification performed autonomously by each slave device (1ₛ). In other words, each slave device (1ₛ) shares with the master device (1ₘ) only the result of the classification performed locally by each slave device (1ₛ) fixed on an anatomical district (AD). Therefore, data sharing involves only the information whether or not at a certain time the district (AD) is affected by a motor deficit.

Anyhow, assessment whether or not the detected anomalies actually correspond to an alarm condition is a task entirely performed by the master device (1ₘ).

The computer program (SW) of the device (1) may have slightly different structure depending on the data sharing level, as it will be explained below by disclosing preferred embodiments. Particularly, as the complexity of the device (2) increases, as well as the volume of shared data, more stringent tolerances on the communication module (14) are imposed to allow communication among the devices (1ₘ, 1ₛ) of the wearable device (2).

In fact, as the volume of shared information increases, the tolerance on time synchronism of the data sent to the master device (1ₘ) from the different slave devices (1ₛ) decreases. By way of explanation and not limitation of the present invention, the synchronization required to the apparatus (2) in the most onerous working conditions does not exceed the second.

### f) Alarm Activation.

In this step of the method according to the present invention, the master device (1ₘ) processes the third dataset (D₃) generated in the previous classification step, and evaluates whether movement anomalies occurred or not. if anomalies actually correspond to an anomalous condition of the person (P), an alarm condition is activated. These tasks may be done concurrently with, or consequently to, the classification performed previously. Assessment whether anomalies actually correspond to an alarm condition is performed by the master device (1ₘ) which acts as a “distributed intelligence” component of the control unit (11).

In one embodiment, the anomaly identification step is performed simultaneously, or almost simultaneously, with the classifying step of the movement sequences extracted from the data flow (SG). In this mode, the master device (1ₘ, 20) immediately activates an alarm signal as soon as it detects a real abnormal condition. Classification, based on normal and abnormal motion patterns (NMP, AMP), by considering only data or classifications (depending on data sharing level and hence whether or not classification is made locally) from a selected number of slave devices (1ₛ), and does not wait for data or classifications from the remaining slave devices (1ₛ).

In an alternative embodiment, identification of anomalies is done after classification of the movement sequences extracted from the data flow (SG) detected by the detectors (10) of the devices (1ₘ, 1ₛ). In this mode, the master device (1ₘ, 20) activates an alarm signal as soon as it completed data classification, or performed an additional classification (depending on data sharing level and hence on whether or not classification is made locally) by considering movement data from all the slave devices (1ₛ).

As mentioned, the master device (1ₘ) evaluates whether anomalies actually correspond to an alarm condition on the basis of the normal and anomalous movement patterns (NMP, AMP) defined during apparatus training in step b). For example, detection of two anomalies by the motion detectors (10) in the left leg and arm almost certainly corresponds to an ischemic attack or a TIA event. To increase the accuracy of the control unit (11) in identifying movement anomalies, it is preferable to share the sampled (D₁) or processed (D₂) or classified (D₃) data by multiple wearable devices (1) positioned on different anatomical districts (AD). Actually, this mode allows false positive minimization.

For example, suppose the person (P) while walking gets a sprained right ankle. The device (1ₛ) positioned on the injured leg records an anomalous movement e.g., a dragging of the injured leg, and hence sends an alarm signal to the master device (1ₘ). In turn, the master device (1ₘ), by analyzing the results of the classifications performed by the other slave devices (1ₛ), does not recognize any anomaly in the movement of the limbs. Thanks to the algorithms (AI) the master device (1ₘ) concludes that the movement of the person (P) does not presents deviations from the normal condition and that the apparent anomaly is actually the result of movements compensation as a consequence of the distortion.

In the method according to this invention an alarm signal via the communication module (14) is sent, if the control unit (11) evaluates with a significant probability that the person (P) is in an anomalous or pathological condition following processing of the third dataset (D₃) generated in the previous classification step.

To be sure that the device (1) of the person (P) is always connected (e.g., internet or telephone network), the connection state is constantly checked by the components of the alarm chain. If a disconnection with the master device (1ₘ, 20) is detected, the processor (12) generates a specific alarm which is sent to both ends of the alarm chain (e.g., family members, doctor, etc.). The alarm can take the form of an audio or light signal emitted by the user interface (15). The steps of the method described above are performed by the wearable apparatus (2) according to the present invention in “real time” mode, i.e., an instant response following an event involving the person (P).

This mode, also named “AI-embedded” or “AI-on-the-edge”, is enabled by the architecture of the algorithm that implements a shared AI between slave/master devices (1ₛ, 1ₘ) with low latency detection. In this case “real time” refers to the fact that execution times of certain modules of code must be known.

For example, the motion sensor signals (10) must be sampled in a time frame that allows sampling at 100 Hz without fluctuations.

For this purpose, the operating system executes several processes simultaneously and sampling has absolute priority among them. For example, when the processor (12) of the device (1ₛ, 1ₘ) execute the classification algorithms (AI), the neural network, as soon as signal sampling is required, interrupts the classification task, performs signal sampling and then returns to execution of the classification algorithms where it left off.

This operating mode is particularly advantageous in may operating conditions. For example, when an elderly person falls, the device (1) is able to recognize the event shortly after, because it locally analyzes the recorded data without having to send them to the master device (1ₘ, 20).

### PREFERRED EMBODIMENTS

### First Preferred Embodiment

With reference to the enclosed FIG. 6, the first preferred embodiment refers to a wearable device (2) configured to detect movement anomalies associated with, or predictive of, a stroke or a TIA event affecting the monitored person (P). The immobility or motor deficit of a limb can be detected by a single device (1) worn on the anatomical district (AD) of the limb itself. This means that by wearing at least one device (1) on each anatomical district (AD) of the upper and lower limbs, it is possible to detect a stroke event. In turn, the detected movement anomalies can be correlated with the cognitive deficits of the person (P), or with a reference statistical sample for that person. It shall be apparent to those skilled in the art that such deficits can be detected by performing tests directly on the person (P), or by using already available medical studies.

Consequently, the apparatus and method according to the present invention advantageously allows to identify cognitive deficits of a person (P) by analyzing movement anomalies of specific anatomical districts (AD).

In the first preferred embodiment, the wearable apparatus (2) includes 4 devices (1) which are positioned on each of the arms and on each of the thighs. Said devices (1) includes each a single sensor (10) in the form of a tri-axial accelerometer, and a wireless communication module (14) of the Bluetooth® Low Energy (BLE) type.

In this preferred embodiment, said devices (1) exchange movement data (SG) acquired by the detectors (10) with a low level of sharing. In this mode, the only information shared between the devices (1) is the classification result of the events (stroke Yes/No) identified when movement data flow processing is ended.

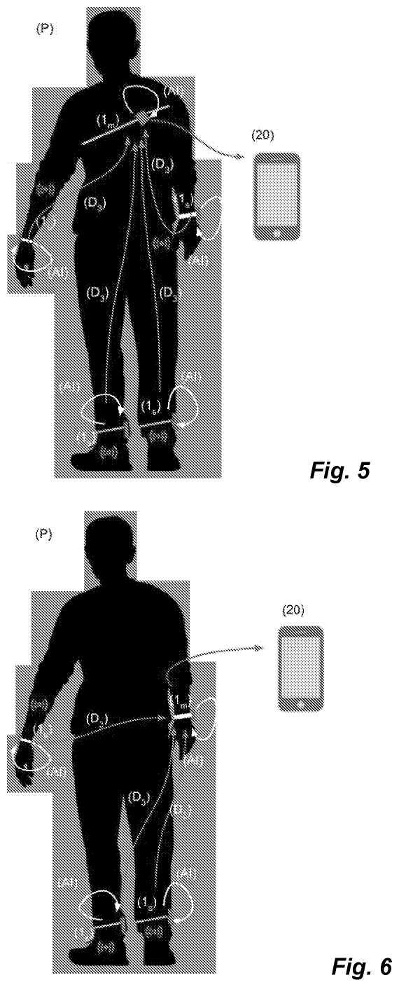

With reference to the enclosed FIG. 5, in the first preferred embodiment, the computer program (SW) implemented by each processor (12) of the devices (1) includes a firmware operating in real time. Said operating mode is described below.

The firmware is configured to manage three states that the device (1) can assume: monitoring state; alarm state; charging state.

When booting, the processor (12) initializes the device (1) in the monitoring state. In this state, three main tasks are running in parallel: measurement, classification, and communication. The measurement task is repeated with absolute priority at a frequency of 100 Hz, thus allowing the three inertial signals (i.e., the accelerations along the three space axes) to be sampled at the same frequency. This information is saved in the main memory (132) of the device (1) where a buffer of samples of adequate size is kept to guarantee functioning of the apparatus (2). The firmware ensures the parallel execution of the second task which, with a period of the order of seconds, classifies the signal and checks whether any anomalous events have occurred (i.e., person falls, motor deficits, fainting, loss of consciousness). Different classification parameters can be applied when the person (P) is asleep. Classification of signal is then stored in the main memory (132) and is then shared with the remaining devices (1ₛ, 1ₘ) of the apparatus (2) worn by the person (P). If signal classification is consistent with a person falling, the processor (12) switches to the alarm state.

While the device (1) remains in the monitoring/measurement state, the processor (12) executes concurrently the communication task, which operates with a lower priority and frequency than the previous two tasks. In this task, each slave device (1ₛ) sends to the master device (1ₘ) the outcome of the local signal classification performed and the battery charge status (161) via the battery manager (162).

In this task transition of the device (1) to the charging state or to the alarm state is also started, respectively, when the processor (12) detects that external power has been applied, or when the battery level drops below the minimum level.

In the first preferred embodiment, described here by way of explanation and not limitation of the present invention, the state transition can also be caused by specific interrupts sent to the processor (12) by the user interface (15), the accelerometer (10) and by the BLE communication module (14).

In particular, the BLE module (14) and the command key of the user interface (15), enable return to the measurement state when the device (1) is in the alarm state (e.g., the accelerometer may trigger the alarm state when a “not worn device” condition is detected). The alarm status consists of a single task: via the BLE communication module (14) a notification with the alarm code is delivered to the master device (1ₘ) which operates properly according to the alarm code received. The master device (1ₘ) enables the device (1) to leave the alarm status only after completion of the tasks required by the alarm code.

To conclude, the charging status consists also of a single task. As mentioned, during monitoring each device (1) collects information on the movements made by the person (P) to be monitored, such as number of steps taken, period spent at rest, etc. This movement data is stored in the memory and a report is generated when the device is charged. The report is sent to the master device (1ₘ) in the charging stage to minimize battery consumption during monitoring and therefore extend its life.

### Second Preferred Embodiment

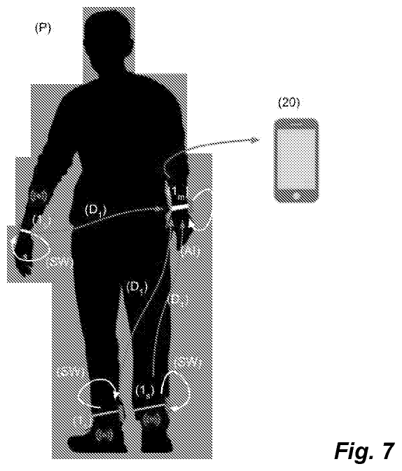

As the enclosed FIG. 7 schematically shows, the second preferred embodiment refers to the same wearable apparatus (2) of the previous embodiment, but differs in the way (i.e., sharing level) the devices (1) share movement data (SG) acquired by the detectors (10).

In the second preferred embodiment the sharing level is partial or total. Compared to the first embodiment, this leads to differences in the task which measures the monitoring status.

In the case the sharing level is partial, data are partially locally processed by a single slave device (1ₛ) and are then sent to the master device (1ₘ) at lower frequencies via the BLE communication module (14).

In the case the sharing level is total, no signal classification is performed by the processor (12) of the single slave device (1ₛ) and data in raw form is sent to the master device (1ₘ) via the Bluetooth® Low Energy communication module (14).

### Third Preferred Embodiment

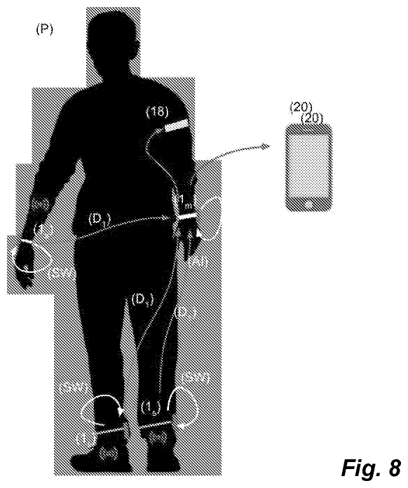

In the third preferred embodiment, herein illustrated in the enclosed FIG. 8, the wearable apparatus (2) is one of the previous embodiments but in addition includes administration means (18) which enables automatic or manual-assisted administration of a drug according to an appropriate therapy protocol.

### Fourth Preferred Embodiment

In the fourth preferred embodiment, the wearable apparatus (2) records the data flow (SG) generated by movement devices (1), analyzes the time frame series and by means of specific AI algorithms try to identify movement patterns associated to a set of specific pre-defined normal actions defining normal movement patterns (NMP). By way of explanation of the invention, and not as a limitation thereof, normal actions include walking, climbing the stairs, sitting down, standing up, but also well-executed movements in sports activity (e.g., of best players or best performance of an athlete), or proper movements during post-trauma motor rehabilitation (e.g., movement to be done to accelerate post-trauma recovery or according to post-trauma motor retraining).

In other words, each time frame is classified against said set (NMP) and if ranked with a score, which expresses the distance respect to the ideal movement, i.e., how good the movement is executed compared to the normal physiological pattern.

A high score is given to the time window if the movement fits well the physiological dataset (NMP) i.e., the movement is well executed, without hesitation and in the correct timing.

Conversely, a low score is given if the pattern of the time window is distant from the physiological pattern (NMP). If the action does not fall within the set, it is discharged.

An overall score is then obtained from a weighted combination of the single movement scores. In case the movement anomalies are related to, of predictive of, a pathology, the overall score is correlated to the risk of fall in the subsequent days or weeks.

In sports practice or post-trauma rehabilitation, the overall score is correlated to athlete or patient improvement.

In this way, the apparatus (2) helps the physician, sports trainer or care-giver by providing an objective and uniform judgment, which is based on objective data derived from personal experience, literature proven experimental clinical protocols, or sports best practices. Assessment of movement anomalies, particularly the fall risk for the patient, is thus automated.

### BIBLIOGRAPHICAL REFERENCES

1. Rokach, L. (2010). “Ensemble-based classifiers”. Artificial intelligence review, 33(1), 1-39.
2. Goodfellow, I., Bengio, Y., Courville, A., & Bengio, Y. (2016). “Deep learning” (Vol. 1, No. 2). Cambridge: MIT press.
3. Hannun, A. Y., Rajpurkar, P., Haghpanahi, M., Tison, G. H., Bourn, C., Turakhia, M. P., & Ng, A. Y. (2019). “Cardiologist—level arrhythmia detection and classification in ambulatory electrocardiograms using a deep neural network”. Nature medicine, 25(1), 65-69.
4. Ho, Tin Kam. “Random decision forests.” Proceedings of 3rd international conference on document analysis and recognition. Vol. 1. IEEE, 1995.
5. Vapnik, Vladimir. “The nature of statistical learning theory”. Springer science & business media, 2013.
6. Hastie, Trevor, Robert Tibshirani, and Jerome Friedman. “The elements of statistical learning: data mining, inference, and prediction”. Springer Science & Business Media, 2009.

### CONCLUSIONS

To conclude, it has been found that the invention described hereinabove fully achieves the intended aim and objects.

The invention is not limited to the exemplary embodiments shown and described herein and although the description and examples provided contain many details, these should not be construed as limiting the scope of the invention but simply as illustrations of some embodiments of the present invention.

For example, the embodiments are mostly referred to an apparatus and a method capable of detecting movement anomalies which are related to, or predictive of, the onset of a pathology causing motor deficits, particularly a TIA or a stroke. However, it is understood that movement anomalies may be related to different pathologies such as epilepsy, Asperger's syndrome, Parkinson's syndrome, autism spectrum disorders.

In addition, it is understood that movement anomalies may be also related with totally different applications such as the practice of sports by athletes, particularly professional athletes, or post-trauma motor rehabilitation.

Hence, any modification of the present invention which falls within the scope of the following claims is considered to be part of the present invention.

Where the characteristics and techniques mentioned in any claim are followed by reference signs, these reference marks have been applied solely for the purpose of increasing the intelligibility of the claims and consequently these reference marks have no limiting effect on the interpretation of each element identified by way of example from these reference signs.

1. A wearable apparatus, for analyzing the movement of a person, the apparatus comprising:
one or more devices for tracking the movement of one or more anatomical districts of a person, wherein said one or more devices each comprise one or more motion detectors which are configured to generate a movement data flow of a speed or acceleration or position of said one or more anatomical districts;
a distributed or centralized control unit in functional connection with said one or more devices or with said one or more devices and with one or more remote electronic devices, said control unit comprising a processor and a memory unit having a computer program stored therein, said computer program being executable by said processor and being configured to process the data flow so as to obtain one or more processed datasets;
a communication module which in association with said control unit, allows data exchange between two or more of said devices, or between at least one of said devices and said remote electronic device, wherein said data are the data flow generated by the motion detectors or said data is said one or more datasets processed by the control unit;
a centralized or distributed power device to power the wearable device;
one or more wearable casings to contain said one or more devices, and at least a part of said communication module;
a user interface which in association with said software allows configuration and functions activation of said wearable device as well as activation of an alarm condition by means of an alarm device,
said wearable apparatus wherein:
one of said one or more devices is a master device and the remaining wearable devices are slave devices; or
said devices are all slave devices and the master device is said remote electronic device,
said master device receives from said slave devices said data flow or said one or more processed datasets, and performs a first classification or a further classification in addition to the classifications performed locally by the individual slave devices,
(wherein said computer program includes one or more artificial intelligence algorithms, which, when executed by the processor of said processing unit, direct said processor to:
identify in said one or more processed datasets, optionally in real time, potential anomalies in the movement of the person with respect to a normal movement pattern according to a paradigm of low-latency detection and
associate to said anomalies the probability that an anomalous event is in progress or may arise in said person.

2. The wearable apparatus according to claim 1, wherein said one or more motion detectors are of the type selected from the group consisting of: an inertial sensor, a gyroscope, an accelerometer, a magnetometer, a camera, and combinations thereof.

3. The wearable apparatus according to claim 1 wherein:
the memory unit comprises a main memory and a mass memory;
the remote electronic device comprises an external memory unit selected from: a smart-phone, a tablet, a personal computer, a gateway, a video game console, or a combination thereof;
the communication module is a Bluetooth® Low Energy communication module.

4. The wearable apparatus according to claim 1, wherein said one or more motion detectors, at least one processing unit and at least a part of the communication module are contained in a wearable enclosure, said enclosure comprising means for retaining said device on an anatomical district.

5. The wearable apparatus according to claim 1, wherein said one or more processed datasets include:
a first dataset obtained by sampling said data flow;
a second dataset obtained by applying a processing operator to said first dataset;
a third dataset obtained by applying said one or more artificial intelligence algorithms to said second dataset.

6. The wearable apparatus according to according to claim 5, wherein the devices exchange data by means of said communication module with one of the following sharing levels:
a total sharing level, when said first dataset is generated by each individual device and is sent to the single master device which processes and classifies said first dataset, so that said second dataset and said third dataset are generated;
a partial sharing level, when each individual slave device generates said second dataset and shares it with the master device;
a minimum sharing level, when each individual slave device generates said second dataset and said third dataset, and shares with the master device only said third dataset i.e. the result of the classification performed independently by each individual slave device.

7. The wearable apparatus according to claim 1, comprising
one or more devices applied to upper limbs of said person; or
one or more devices applied to lower limbs of said person; or
one or more devices applied on the head, or neck, or on the trunk or pelvis.

8. The wearable apparatus according to claim 1, further comprising an administration means configured to automatically or manually administer a compound if an anomalous event is in progress or may arise in said person.

9. The wearable apparatus according to claim 1, for use in the analysis of anomalies affecting the movement of a person, wherein said anomalies are predictive of, or related with, a human pathological condition associated with features which are detectable by the movement of one or more anatomical districts of said person.

10. The wearable apparatus according to claim 9 wherein said pathological condition is selected from: Transitory Ischemic Attack, stroke, epilepsy, Asperger's syndrome, Parkinson's syndrome, autism spectrum disorders, or a combination thereof.

11. A method for a movement analysis of a person by means of the wearable apparatus according to claim 5, said method comprising the following steps:
a) choosing a wearable apparatus having one or more devices each including one or more motion detectors in functional connection with a centralized or distributed control unit, said one or more devices positioned on at least one anatomical district of said person for tracking movement thereof when said person moves;
b) initializing and training said wearable apparatus by providing an ensemble of one or more classification algorithms with a set of normal movement patterns of the person, or with a set of abnormal movement patterns associated with abnormal movement of the person, said classification algorithms being included in the software of said control unit;
c) acquiring the movement data flow of the person generated by said motion detectors and sample said flow so as to generate a first dataset;
d) applying a processing operator to said first dataset so as to obtain a second dataset, said operator being at least one of:
a time synchronization operator if said first dataset consists of signals generated from different devices,
an operator able to obtain a representation of said first dataset, in the form of a features vector;
an operator able to extract useful information by reducing the flow of data on the transmission channels.
e) classifying said second dataset by applying one or more artificial intelligence algorithms in order to generate a third dataset which expresses the degree of similarity of the movement of the person, associated with the data flow detected in step c), with respect to said set of normal movement patterns or said set of anomalous movement patterns, wherein the classification is:
performed independently by each individual slave device only on the signals collected, sampled and processed by said slave device, and therefore only on elements of the first dataset or of the second dataset, or
performed by the master device on all the signals collected by the individual slave devices, and therefore on the entire first dataset or on the entire second dataset;
f) evaluating, upon processing said third dataset, the likelihood of a real anomaly in the movement of said person associated with the data flow detected in step c), and activate an alarm signal by means of said control unit according to an outcome of the likelihood evaluation.

12. The method according to claim 11 wherein a sharing level between the devices is:
a total sharing level, when said first dataset is generated by each individual device and is sent to the single master device which processes and classifies said first dataset, so that said second dataset and said third dataset are generated;
a partial sharing level, when each individual slave device generates said second dataset and shares it with the master device;
a minimum sharing level, when each individual slave device generates said second dataset and said third dataset, and said slave device shares with the master device only said third dataset being the result of the classification performed independently by each individual slave device.

13. The method according to claim 11, wherein said ensemble of classifiers are selected from the group consisting of: recurrent neural networks, convolutional networks, one-dimensional convolutional neural networks, random forests, support vector machines, k-nearest neighbor, and a combination thereof.

14. The method according to claim 11, wherein the movement analysis of a person is addressed to detect one or more anomalies in the movement which are predictive of, or related with, a cognitive deficit or a pathology or post-traumatic condition, a Transitory Ischemic Attack event, stroke, epilepsy, Asperger syndrome, Parkinson's syndrome, autism spectrum disorder, or a combination thereof.

15. The method according to claim 11, wherein the analysis of the movement is addressed to optimize the sports performance of an athlete.
